(19) United States
(12) Patent Application Publication
(10) Pub. No.: US 2011/0178604 A1
(43) Pub. Date: Jul. 21, 2011

(54) Expandable Endoprosthesis

(75) Inventor: Joshua R. Porter, Winona Lake, IN (US)

(73) Assignee: Biomet Manufacturing Corp., Warsaw, IN (US)

(21) Appl. No.: 12/689,115

(22) Filed: Jan. 18, 2010

(51) Int. Cl. A61F 2/40; A61F 2/28; A61F 2/38

(52) U.S. Cl. 623/19.14; 623/23.47; 623/20.32; 623/20.35

(57) ABSTRACT

An implantable device includes a first implant component implantable into a first bone portion, a second implant component implantable to a second bone portion and a third implant component comprising a shape-memory polymer. The second implant component is telescopically coupled to the first component. The third implant component is held between the first and second components. The third implant component passively expands from a first dimension to a second dimension during a selected time interval after implantation.

### INTRODUCTION

Various endoprosthesis components include expandable elements activated by external means, including mechanical, hydraulic or magnetic activators and combinations thereof.

The present teachings are directed to various expandable endoprostheses with passive activation.

### SUMMARY

The present teachings provide an implantable device that includes a first implant component implantable into a first bone portion, a second implant component implantable to a second bone portion and a third implant component comprising a shape-memory polymer. The second implant component is telescopically coupled to the first component. The third implant component is held between the first and second components. The third implant component passively expands from a first dimension to a second dimension during a selected time interval after implantation.

The present teachings provide an implantable device including first and second implant components implantable into bone. The first and second implant components are coupled for telescopic movement relative to one another along a longitudinal axis of the implantable device. A third implant component is positioned between the first and second components along the longitudinal axis. The third implant component has a first length before implantation at a first time, and expands after implantation along the longitudinal axis to a second length greater than the first length at a second time different than the first time, such that the implantable device expands by an amount equal to the difference between the first and second lengths.

The present teachings provide a method for expanding an implantable device. The method includes implanting first and second implant components into bone and telescopically coupling the first and second implant components along a longitudinal axis. The method also includes providing a third implant component comprising a shape memory polymer having a first length before implantation. The third implant component is positioned between the first and second implant components along the longitudinal axis. The third implant component passively expands after implantation to a second length over a selected period of time. The implantable device passively expands by an amount equal to the difference between the second and first lengths by a corresponding expansion of the third implant component.

Further areas of applicability of the present teachings will become apparent from the description provided hereinafter. It should be understood that the description and specific examples are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1A is a plan view of an exemplary expandable endoprosthesis according to the present teachings;

FIG. 1B is a plan view of the expandable endoprosthesis of FIG. 1A illustrated after in situ expansion;

FIG. 1C is schematic diagram illustrating expansion length as a function of time for an exemplary shape memory polymer used in a plug of the expandable endoprosthesis of FIG. 1;

FIG. 2 is a plan view of an exemplary expandable endoprosthesis according to the present teachings;

FIG. 3 is an exploded view of an exemplary expandable endoprosthesis according to the present teachings;

FIG. 4 is an environmental view of an exemplary expandable endoprosthesis according to the present teachings;

FIG. 5A is an exploded plan view of an exemplary expandable endoprosthesis according to the present teachings; and

FIG. 5B is an assembled plan view of the expandable endoprosthesis of FIG. 5A.

### DESCRIPTION OF VARIOUS ASPECTS

The following description is merely exemplary in nature and is in no way intended to limit the present teachings, applications, or uses. For example, although the present teachings are illustrated for applications in long bones, such as the proximal humeral bone and the proximal and distal femoral bones in knee surgery, the present teachings can be used any other expandable endoprostheses in which passive expansion is desirable. Further, the present teachings can be used in applications in connection with total joint replacement, revision surgery, trauma, limb salvage surgery, distraction osteogenesis, pediatric procedures, cancer-related procedures, such as those related to osteosarcoma.

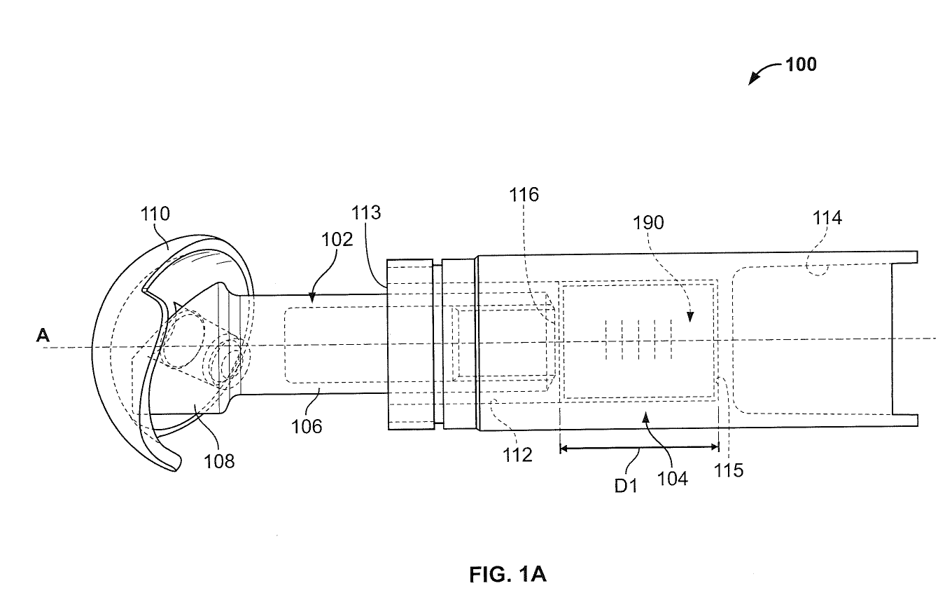

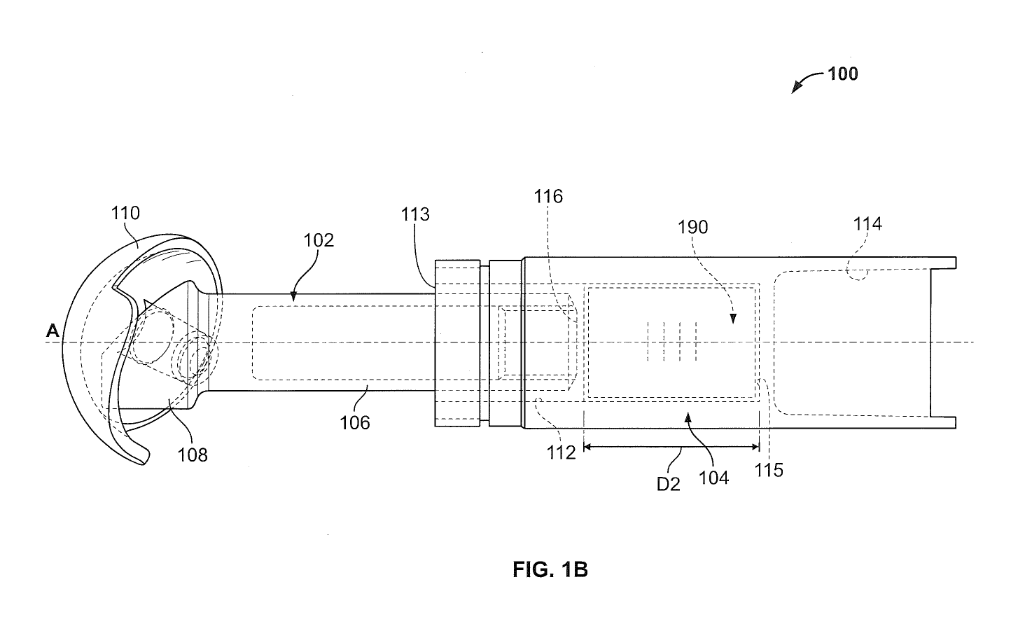

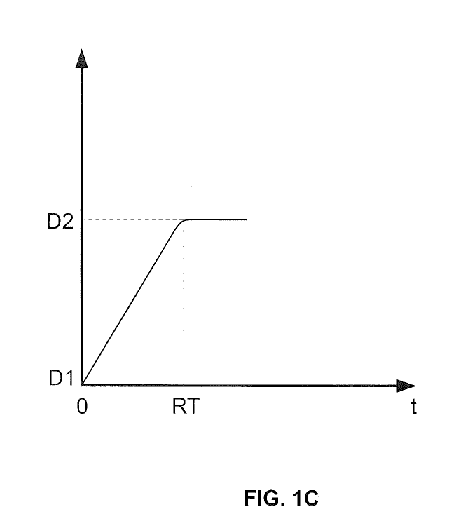

Referring to FIG. 1A, an exemplary implantable device or expandable endoprosthesis 100 for a proximal humeral bone according to the present teachings is illustrated. The expandable endoprosthesis 100 can include a proximal or first implant component 102 and a distal or second implant component 104. The first and second implant components 102, 104 can be connected to one another for relative axial telescopic movement along a longitudinal axis A of the expandable endoprosthesis 100. In the exemplary embodiment of FIG. 1, the first implant component 102 can include a body portion 108 and a shaft portion 106 extending from the body portion 108 to a distal end 116. The body portion 108 can be modularly/removably or fixedly attached to a head 110, which can articulate with the glenoid cavity of the scapula. The second implant component 104 can include a longitudinal blind bore or an expansion chamber 112, which receives a portion of the shaft 106 for telescopic movement. The blind bore 112 extends from an open proximal end 113 to a closed distal end 115. The second implant component 104 can also include a distal cavity 114 for modular connection to a stem component or other distal component of the modular implant 100 for anchoring into the bone.

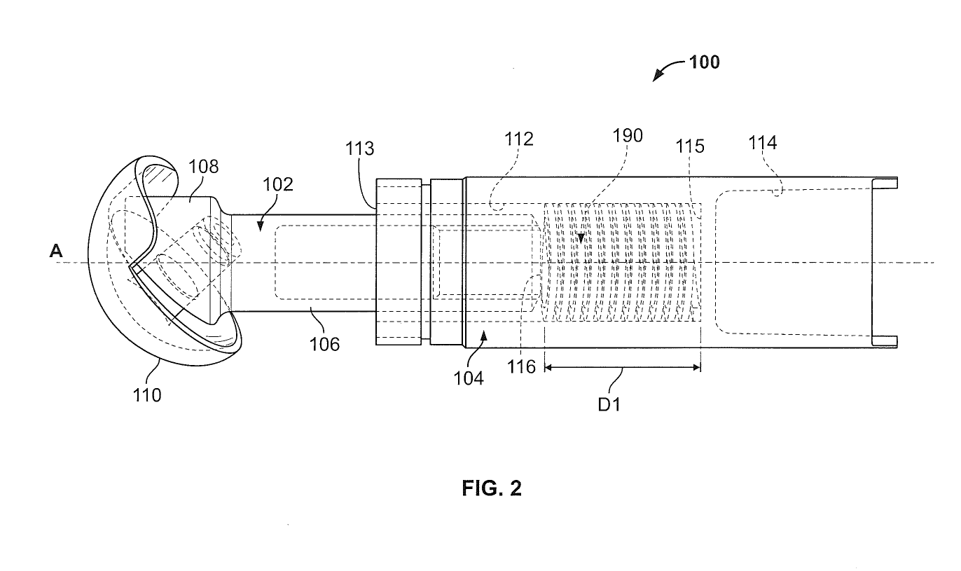

Referring to FIGS. 1A, 1B and 2, a plug 190 is received in the blind bore 112 between the distal end 116 of the shaft 106 and the distal end 115 of the blind bore 112. The plug 190 can include a shape-memory polymer (SMP) of the type disclosed in US Patent Application Publication 2009/0248141 (the “'141 publication”), which is incorporated by reference herein. The materials and processing of the plug 190 can be selected for gradual passive activation upon implantation at body temperature. In particular, the SMP can include a plurality of monomers, such as a first selected monomer and a second selected cross-linking monomer with selected weight percentages. The material of the plug 190 can include, in addition to the SMP, other non shape-memory materials, including reinforcing fibers or other materials. The plug material can be processed at a selected glass transition temperature, with selected cross-linking and other processing, such that upon implantation the plug 190 expands passively from a first predetermined length D₁ to a second predetermined length D₂, which is greater than D₁, during a predetermined recovery time (RT), as illustrated schematically in FIG. 10. Passive expansion is defined as expansion that does not require any external activation means either in the form of mechanical/invasive means or in the form of noninvasive remotely applied activation means, such as, for example, electromagnetic, ultrasound, radiation or heating agents that that cause the shape memory polymer of the plug 190 to reach a glass transition or other transition state triggering expansion from a pre-implantation length to a new length. Accordingly, passive expansion occurs by selecting a transition temperature for the shape memory polymer of the plug 190 such that the change in length is initiated when the plug 190 reaches an implantation or body temperature of the anatomic site, such a humeral, femoral or tibial bone, when the expandable endoprosthesis is used in orthopedic applications.

Referring to FIG. 10, the slope of the illustrated length versus time (t) graph represents the expansion rate, which can also be selected. A constant expansion rate is illustrated in FIG. 1C, as indicated by the straight line, although other expansion rates, such as non-linear or stepwise linear can be selected by changing the various chemical, physical, mechanical or other properties of the SMP material of the plug 190. Various SMP materials, processes, manufacturing methods for structural elements made thereof and related structures can be found in the '141 publication. Additional information related to SMP materials can be found in Yakacki et al, “Unconstrained recovery Characterization of shape-memory polymer networks for cardiovascular applications,” Biomaterials, Vol. 28, pp. 2255-2263, 2007; and in Bellin et al, “Polymeric triple-shape materials,” PNAS, Vol. 103, No. 48, pp. 18043-18047, 2006, each of which are incorporated by reference herein.

The plug 190 can be formed as a solid cylindrical element, as illustrated in FIG. 1A, or as a coil or other spring like structure, as illustrated in FIG. 2. Other shapes can also be used, including fenestrated, ribbed, hollow plug shapes, and closed or open or semi-open tubular shapes, tubular shapes having a plurality of through apertures of perforations, or other three-dimensional networks of linear of curved elements. Further, the plug 190 can be in the form of a plurality of separate and discrete segments or interconnected segments. The plug segments can have variable properties including variable expansion rates, recovery times and expansion dimensions. Various exemplary shapes are illustrated in the '141 publication.

After implantation of the expandable endoprosthesis 100, the plug 190 can gradually expand from an initial first length D₁ to a second length D₂, over the passage of an interval of time RT. Accordingly, the expandable endoprosthesis gradually changes length over the interval RT by a total amount d=(D₂−D₁) along the longitudinal axis A of the expandable endoprosthesis. The change in length d can be selected to either mirror or cause a corresponding lengthening of the anatomic site, such as a long bone, for example, in which the expandable endoprosthesis 100 is implanted. Alternatively or additionally, the change in length d can provide a dynamic load at the anatomic site for bone growth or bone maintenance, for example. The expandable endoprosthesis 100 can be used for various therapeutic, restoration, correction, salvage or other procedures for providing controlled expansion over a certain interval of time.

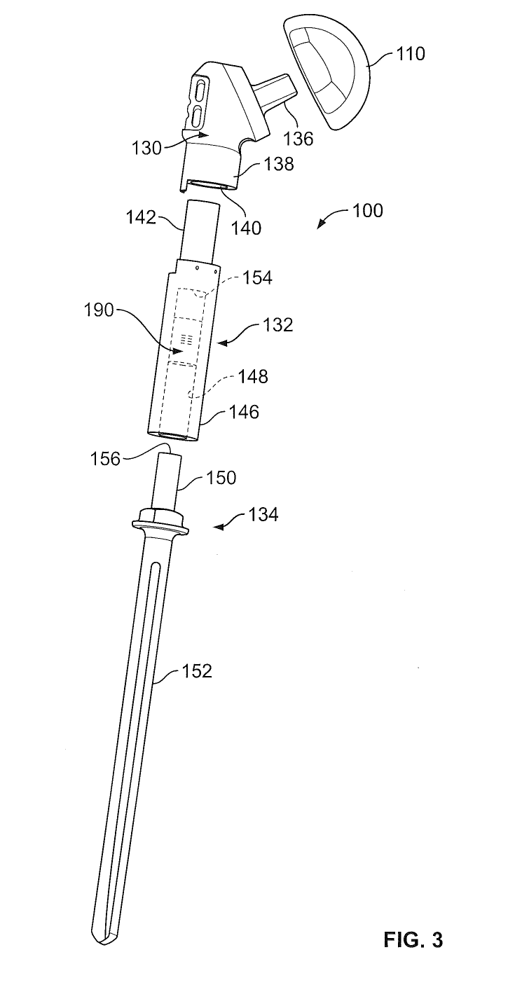

Referring to FIG. 3, another exemplary expandable endoprosthesis 100 according to the present teachings is illustrated for the humeral bone. The expandable endoprosthesis 100 includes a plurality of modularly connected components, including a head 110, a body 130, a proximal shaft 132 and a distal stem 152. The body 130 can include a proximal extension 136 couplable with the head 110 and a distal extension 138 having an inner bore 140 couplable with a proximal extension 142 of the shaft 132. The connections of the body 130 with the head 110 and the shaft 132 can be taper lock connections.

With continued reference to FIG. 3, the shaft 132 can include an inner blind bore/expansion chamber 148 extending from a proximal closed end 154 to a distal open end 146. The plug 190 can be received in the bore 148. The stem 134 can include a proximal cylindrical extension 150 telescopically received within the blind bore/expansion chamber 148 for relative axial movement therein and a distal portion 152 for anchoring in the bone. The plug 190 can operate as described above in connection with FIGS. 1A-C and 2. Specifically, the plug 190 is made so that it can change length by a predetermined/selected amount d during a predetermined recovery time RT, such that the expandable endoprosthesis 100 expands by a distance d along the longitudinal axis A as selected for various therapeutic applications, as discussed above.

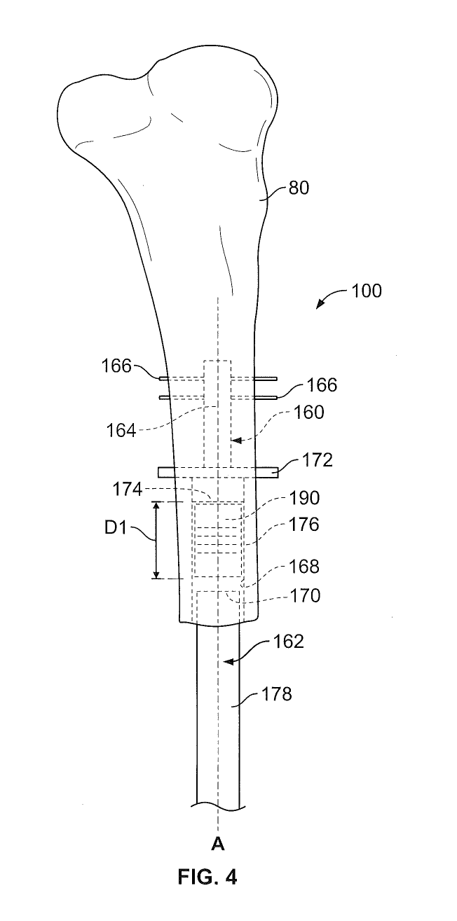

Referring to FIG. 4, an exemplary expandable endoprosthesis 100 according to the present teachings is illustrated for the distal femur. The expandable endoprosthesis 100 includes first and second components 160, 162 telescopically coupled. The first component 160 can include a tubular portion 176 defining a blind bore/expansion chamber 168 and an anchoring portion 160. The anchoring portion 160 can include a proximal shaft 164 that can be anchored to the bone 80 with transverse cross pins 166 and or a plate 172. The blind bore 168 can have a closed end 174. The second component 162 can include a proximal cylindrical shaft 178, which is telescopically received into and axially movable relative to the blind bore 168. The plug 190 is received between the closed end 174 of the blind bore 168 and a proximal end 170 of the proximal shaft 178 of the second component 162. As discussed above, the plug 190 is made so that it can change length by a predetermined amount d during a predetermined recovery time RT, such that the expandable endoprosthesis 100 expands along the longitudinal axis A by a distance d as selected for various therapeutic applications.

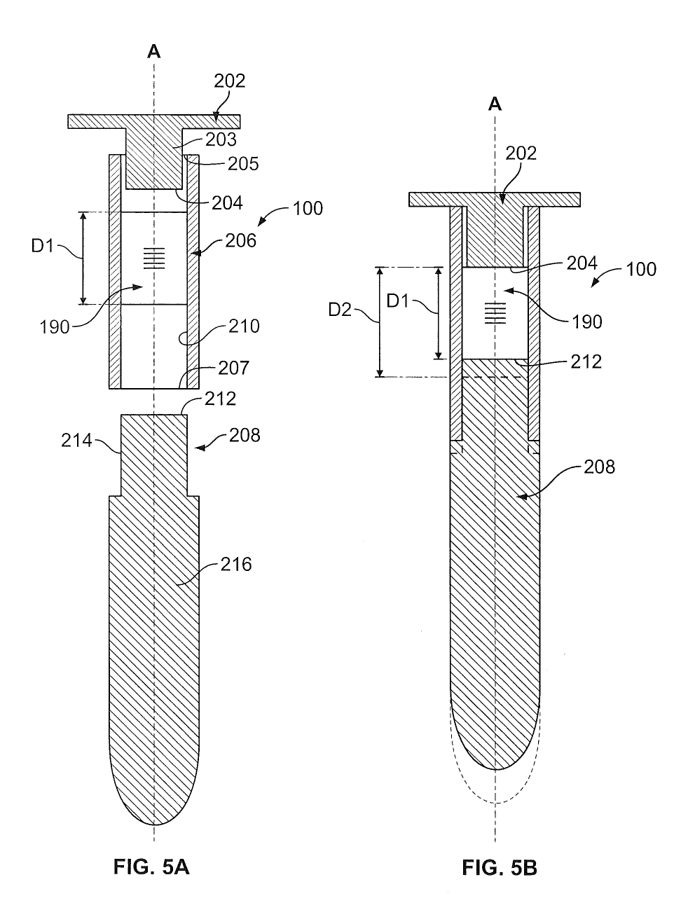

Referring to FIGS. 5A and 5B, an exemplary expandable endoprosthesis 100 according to the present teachings is illustrated for the tibia. In this exemplary embodiment, the endoprosthesis includes a tibial tray 202, an intermediate component 206 and a stem 216. The intermediate component 206 can be a hollow shaft including an inner bore or expansion chamber 210 with first and second open ends 205, 207. The tibial tray 202 can include an extension 205 with a distal end 204. The extension 205 can be coupled to the first end 203 of the bore 210 with a taper lock connection, for example, as illustrated. The stem 208 can include an anchoring portion 216 and a cylindrical extension 214 with a proximal end 212. The extension 214 can be telescopically received into and axially movable relative to the bore 210. The plug 190 can be received in the bore 210 between the distal end 204 of the extension 205 of the tibial tray 202 and the proximal end 212 of the extension 214 of the stem 208. As discussed above, the plug 190 is made so that it can change length by a predetermined amount d=(D₂−D₁) during a predetermined recovery time RT, such that the expandable endoprosthesis 100 expands by a distance d along the longitudinal axis A as selected for various therapeutic applications.

As discussed above, the present teachings can be applied to various procedures in which bone lengthening is anticipated or is desirable. Examples include limb salvage procedures in pediatric patients, in which a resected bone segment can be replaced with a modular prosthetic device which can be lengthened after implantation by incorporating an SMP plug 190 as described above. Further, the present teachings provide passive activation of the SMP plug 190 upon implantation over a predetermined period of time RT by controlling the chemical, physical and other properties of the plug 190.

As discussed above, the present teachings utilize an SMP plug 190 positioned between first and second telescopically connected components to achieve a selected amount of lengthening of the expandable endoprosthesis 100 at a selected recovery time passively, i.e., without requiring activation for expansion. Accordingly, invasive activation means such as gear or screw driven expansion is avoided. Further, non-invasive activation means, such as magnetically driven expansion is also avoided. The passive expansion of the expandable endoprosthesis 100 according to the present teachings can be selected to either accommodate or follow a natural bone or limb lengthening. Accordingly, the risk, inconvenience and discomfort associated with invasive procedures can be avoided. Similarly, pain, discomfort and potential tissue damage associated with active expansion by non-invasive activation is also avoided.

It should appreciated that the expandable endoprosthesis 100 is anchored to the patient's anatomy. The SMP plug 190 is loosely or freely retained in a bore or expansion chamber of the endoprosthesis 100 and can expand according to a predetermined time schedule that follows a corresponding expansion/growth of the patient's anatomy. Alternatively, the SMP plug 190 can provide a dynamic load against a fixed-length anatomy of the patient.

The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the present teachings without departing from the essential scope thereof. One skilled in the art will readily recognize from such discussion, and from the accompanying drawings and claims, that various changes, modifications and variations can be made therein without departing from the spirit and scope of the present teachings as defined in the following claims.

What is claimed is:

1. An implantable device comprising:
a first implant component implantable into a first bone portion;
a second implant component implantable to a second bone portion, the second implant component telescopically coupled to the first component; and
a third implant component comprising a shape-memory polymer, the third implant component held between the first and second components, the third implant component passively expanding from a first dimension to a second dimension during a selected time interval after implantation.

2. The implantable device of claim 1, wherein the first implant component is telescopically movable relative to the second implant component.

3. The implantable device of claim 2, wherein the second implant component defines a blind bore having a closed end and the first component includes an extension having a distal end, the extension movably received in the blind bore.

4. The implantable device of claim 3, wherein the third implant component is received in the blind bore between the distal end of the extension and the closed end of the blind bore.

5. The implantable device of claim 1, wherein the third implant component comprises a solid plug of a shape-memory polymer.

6. The implantable device of claim 1, wherein the third implant component comprises a coil of shape-memory polymer.

7. The implantable device of claim 1, wherein the first implant component includes a tibial tray coupled to a tubular shaft and the second implant component includes a tibial stem telescopically coupled to the tubular shaft.

8. The implantable device of claim 7, wherein the tibial tray is taper locked to the tubular shaft.

9. The implantable device of claim 1, wherein the third implant component expands from the first dimension to the second dimension at a selected expansion rate.

10. The implantable device of claim 1, wherein the first and second implant components comprise components of an orthopedic implant selected from a group of humeral, femoral and tibial implants.

11. An implantable device comprising:
first and second implant components implantable into bone, the first and second implant components coupled for telescopic movement relative to one another along a longitudinal axis of the implantable device;
a third implant component positioned between the first and second components along the longitudinal axis, the third implant component having a first length before implantation at a first time, the third implant component expanding after implantation along the longitudinal axis to a second length greater than the first length at a second time different that the first time, such that the implantable device expands by an amount equal to the difference between the first and second lengths.

12. The implantable device of claim 11, wherein the third implant component comprises a shape-memory polymer having a selected recovery time and a selected expansion rate.

13. The implantable device of claim 11, wherein the third implant component is received within a blind bore of one of the first and second implant components.

14. The implantable device of claim 11, wherein the first and second implant components comprise corresponding components of a humeral implant.

15. The implantable device of claim 11, wherein the first and second implant components comprise corresponding components of a tibial implant.

16. The implantable device of claim 11, wherein the first and second implant components comprise corresponding components of a femoral implant.

17. A method for expanding an implantable device, the method comprising:
implanting first and second implant components into bone;
telescopically coupling the first and second implant components along a longitudinal axis;
providing a third implant component comprising a shape memory polymer having a first length before implantation;
positioning the third implant component between the first and second implant components along the longitudinal axis, wherein the third implant component passively expands after implantation to a second length over a selected recovery time; and
passively expanding the implantable device by an amount equal to the difference between the second and first lengths by a corresponding expansion of the third implant component.

18. The method of claim 17, wherein telescopically coupling the first and second implant components along a longitudinal axis includes inserting an extension of one of the first and second implant components into a bore of the other of the first and second implant components.

19. The method of claim 18, wherein positioning the third implant component between the first and second implant components along the longitudinal axis includes inserting the third implant component in the bore of one of the first and second implants components.

20. The method of claim 17, wherein passively expanding the implantable device includes passively expanding the implantable device by a selected expansion rate.

21. The method of claim 17, wherein the first and second implant components comprise components of an orthopedic implant selected from a group comprising humeral, femoral and tibial implants.
